(19) United States
(12) Patent Application Publication
(10) Pub. No.: US 2022/0262078 A1
(43) Pub. Date: Aug. 18, 2022

(54) REMOTE DEVICE PROVISIONING AND REMOTE SUPPORT USING AUGMENTED REALITY

(71) Applicant: BROADSTONE TECHNOLOGIES, LLC, Frederick, MD (US)

(72) Inventors: Taimur Aslam, Frederick, MD (US); Andrew J. Surwilo, Allendale, NJ (US)

(21) Appl. No.: 17/650,791

(22) Filed: Feb. 11, 2022

Related U.S. Application Data
(60) Provisional application No. 63/200,107, filed on Feb. 14, 2021.

(51) Int. Cl. G06T 19/00; G02B 27/01

(52) U.S. Cl. CPC G06T 19/006; G02B 2027/014; G02B 27/017

(57) ABSTRACT

According to some embodiments, a method is performed by an augmented reality (AR) device. The AR device comprises a display configured to overlay virtual objects onto a field of view of a user in real-time. The method comprises: establishing an audio-video connection between a first user and a second user; receiving an indication from an AR engine that an AR model from the first user is available to the second user; retrieving the AR model from the AR engine, wherein the AR model represents a real world object in the field of view of the second user; determining a surface in the field of view of the second user for projection of the AR model; and displaying on the determined surface an AR projection based on the AR model to the second user via the display.

### RELATED APPLICATION

This application claims the benefit of U.S. Provisional Patent Application No. 63/200,107 filed on Feb. 14, 2021, the disclosure of which is incorporated herein by reference in its entirety.

### BACKGROUND

The present disclosure relates generally augmented reality (AR), and more particularly to remote device provisioning and remote support using AR.

### SUMMARY

According to some embodiments, a system for performing remote instruction in an augmented reality (AR) environment comprises an AR engine and an AR device. The AR engine comprises one or more processors operable to: establish an audio-video connection between a first user and a second user; determine an AR device associated with the second user; and transmit an indication to the AR device that the AR model is available to the second user. The AR device comprises a display configured to overlay virtual objects onto a field of view of the user in real-time and one or more processors coupled to the display. The one or more processors are operable to: receive the indication that the AR model is available to the second user; retrieve the AR model from the A R engine; determine a surface in the field of view of the second user for projection of the AR model; and display on the determined surface an AR projection based on the AR model to the second user via the display.

In particular embodiments, the AR device is further operable to: receive input from the first user to manipulate the AR model; and manipulate the AR model according to the received input. Similarly, the AR device is further operable to: receive input from the second user to manipulate the AR model; and manipulate the AR model according to the received input. The AR device may be further operable to transmit the manipulations performed on the AR model by the second user to the first user.

In particular embodiments, the AR model represents a real world object and the AR device is further operable to receive audio-video instructions over the audio-video connection for the second user to manipulate the real world object.

In particular embodiments, the AR engine is further operable to store manipulations performed on the AR model.

In particular embodiments, the first user comprises a medical expert and the second user comprises an emergency medical technician (EMT). The first user may comprise an expert repairman for a device and the second user may comprise an owner of the device.

### BRIEF DESCRIPTION OF THE DRAWINGS

FIG. 1 illustrates a block diagram of a system for remote device provisioning and remote support using augmented reality (AR), in accordance with a particular embodiments.

FIG. 2 illustrates a system for remote device provisioning and remote support using AR in operation, according to particular embodiments.

FIG. 3 illustrates a flowchart of a method performed by an AR device, in accordance with particular embodiments.

FIG. 4 is a block diagram illustrating an example AR device.

FIG. 5 illustrates an example of an apparatus to implement one or more example embodiments described herein.

### DETAILED DESCRIPTION OF DISCLOSED EMBODIMENTS

As will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely in hardware, entirely in software (including firmware, resident software, micro-code, etc.) or combining software and hardware implementation that may all generally be referred to herein as a “circuit,” “module,” “component,” or “system.” Moreover, any functionality described herein may be accomplished using hardware only, software only, or a combination of hardware and software in any module, component or system described herein. Furthermore, aspects of the present disclosure may take the form of a computer program product embodied in one or more computer readable media having computer readable program code embodied thereon.

Any combination of one or more computer readable media may be utilized. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable. RF, etc., or any suitable combination of the foregoing.

Computer program code for carrying out operations for aspects of the present disclosure may be written in any combination of one or more programming languages, including a symbolic programming language such as Assembler, an object oriented programming language, such as JAVA®, SCALA®, SMALLTALK®, EIFFEL®, JADE®, EMERALD®, C++, C#, VB.NET, PYTHON® or the like, conventional procedural programming languages, such as the “C” programming language, VISUAL BASIC®, FORTRAN® 2003, Perl, COBOL 2002, PHP, ABAP®, dynamic programming languages such as PYTHON®, RUBY® and Groovy. or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatuses (systems) and computer program products according to aspects of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

Highly trained technicians are in short supply and companies often face the challenge of having a skilled technician available to respond to a customer's needs. The skilled technician has to be dispatched to the customer's site to diagnose and rectify the problem. If a technician is not immediately available, the customer continues to face the problem. Depending on the severity of the problem, the impact can range from a minor annoyance to loss of revenue.

Thus, providing remote support often required that a skilled technician be dispatched to a customer's site to diagnose and rectify the problem. This solution is constrained by the availability of a skilled technician as well as the time required to travel to the customer site. The customer also incurs losses during this period due to the unavailability of the machine or device under repair.

Particular embodiments described herein enable a skilled technician to provide instructions to a remote semi-skilled technician using a multitude of technologies including augmented reality (AR), audio and video conferencing using a TCP/IP broadband network, a 4G/5G cellular network, or satellite broadband network.

The skilled technician using particular embodiments is able to project a three-dimensional model of the item to be fixed. Using augmented reality, the skilled technician may annotate the areas that need to be fixed and explain the repair process to the remote technician. The remote technician does not need to have the same skill level as the instructing technician. Using the three dimensional and augmented reality model, the two technicians may collaborate and rectify the problem.

Particular embodiments supplement audio and video conversations between two technicians with an accurate three-dimensional augmented reality model that can be, used to mark and annotate with descriptive text and markings indicating the affected areas to be fixed, rotate along the X, Y, or Z axes, and/or resize to make the model bigger or smaller in size.

In some embodiments, the augmented reality model may be developed using a computer aided design (CAD) model of the device. Particular embodiments may execute on a computer tablet or smartphone to support a wide range of industries. Some embodiments may be developed for specialized hardware to enable, for example, hands free operation by the remote technician.

Particular embodiments may provide advantages to companies that provide after-sales support to its customers. The services may range from installation support, troubleshooting when a problem arises, or general maintenance at periodic intervals or on-demand. Field technicians and skilled technicians that do not have to travel to a remote customer site may use particular embodiments.

Semi-skilled field technicians may use particular embodiments to service and rectify problems in the field. This reduces the mean time to fix when an issue arises at a customer site, thereby reducing the downtime that a customer incurs, which normally results in a loss of revenue. Some embodiments reduce the cost for field servicing because a skilled technician does not have to be dispatched to a customer site.

Particular embodiments include a software application executable on a computer tablet, smartphone, AR device, or specialized hardware. In some embodiments, the software application enables a three-dimensional CAD object to be converted to a specialized binary representation that can be manipulated and projected to display a model of the object that a company wants to service or repair. The communication between the remote technician and a skilled technician, who is knowledgeable about the object being repaired, may be facilitated using TCP/IP broadband, 4G/5G cellular networks, and satellite broadband networks.

Particular embodiments are described more fully with reference to the accompanying drawings. Other embodiments, however, are contained within the scope of the subject matter disclosed herein, the disclosed subject matter should not be construed as limited to only the embodiments set forth herein; rather, these embodiments are provided by way of example to convey the scope of the subject matter to those skilled in the art.

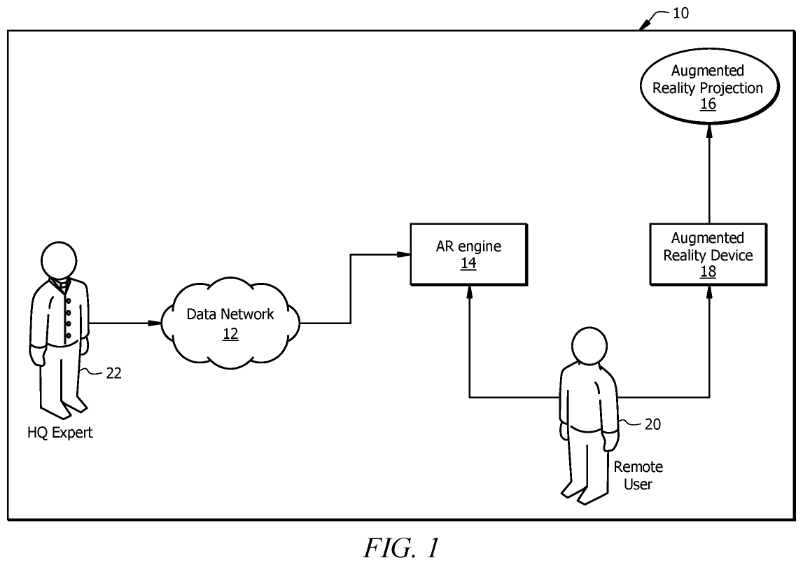

FIG. 1 illustrates a block diagram of a system for e remote device provisioning and remote support (i.e., collectively referred to as remote instruction) using augmented reality (AR), in accordance with a particular embodiment. System 10 includes data network 12. Data network 12 comprises a plurality of network nodes configured to communicate data between the components illustrated in FIG. 1.

Examples of network nodes include, but are not limited to, routers, switches, modems, web clients, and web servers. Data network 12 comprises any suitable type of wireless and/or wired network including, but not limited to, all or a portion of the Internet, the public switched telephone network, a cellular network (e.g., 4G/5G), and/or a satellite network. Data network 12 is configured to support any suitable communication protocols (e.g., TCP/UDP/IP) as would be appreciated by one of ordinary skill in the art upon viewing this disclosure.

System 10 includes AR engine 14. AR engine 14 may comprise an audio/video teleconferencing system. The audio/video teleconferencing system comprises a system that enables audio and video communication between remote technician 20 and expert 22 located at headquarters or some other location separate from remote technician 20. The audio/video teleconferencing system transmits data over data network 12.

AR engine 14 comprises an AR system for transmitting and/or receiving augmented reality models with AR device 18. AR engine 14 my receive input for manipulating an augmented reality model and forward the input to AR device 18. AR engine 14 may receive input reflecting manipulations performed on augmented reality model by AR device 18.

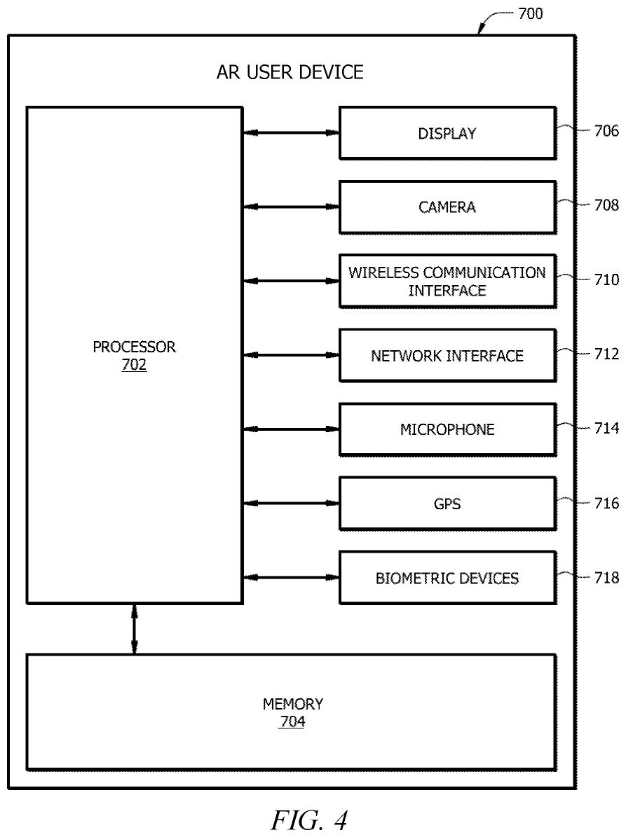

System 10 includes AR device 18. AR device 18 renders, processes, and displays augmented reality model. AR device 18 may comprise, for example, a smart phone, smart glasses, head mounted visor, computer tablet, desktop computer, etc. AR device 18 is described in more detail with respect to FIG. 4.

In particular embodiments, AR device 18 determines a surface in the field of view of a user for projection of the AR model. In some embodiments, AR device 18 may transmit an image of the field of view of remote technician 20 to AR engine 14. AR engine 14 may analyze the field of view and identify a suitable surface for projection of the AR model. In some embodiments, AR device 18 itself may analyze the field of view and identify a suitable surface.

Also included in system 10 is augmented reality projection 16. Augmented reality projection 16 results from the processing and display made possible by AR device 18. Augmented reality projection 16 may, for example, represent a real world object such as a machine or device under repair. The AR projection includes instructional information regarding the real world object.

Remote technician 20 and/or expert 22 interact with augmented reality projection 16 by performing pan, rotate, zoom in, zoom out, and flip operations. These operations may be stored in a database for later review.

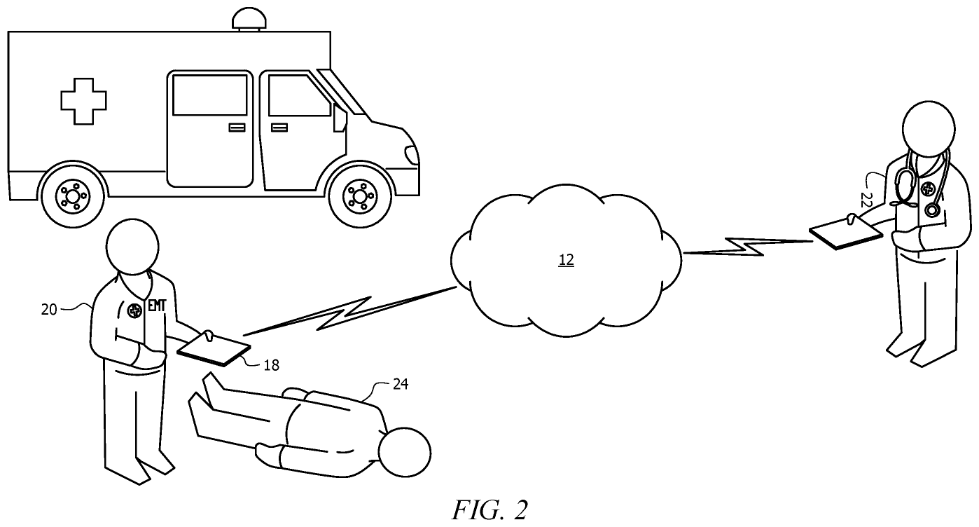

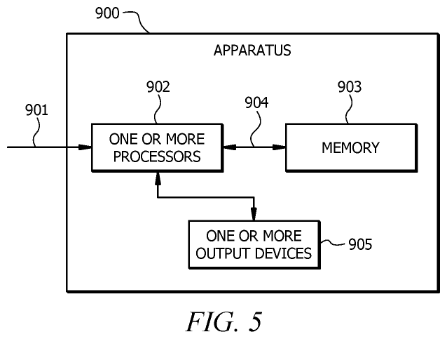

Some components of system 10 engine may be implemented according to one or more of the apparatus described with respect to FIG. 5. An example of system 10 in operation is described with respect to FIG. 2.

FIG. 2 illustrates a system for remote device provisioning and remote support using AR in operation, according to particular embodiments. In the illustrated example, the remote support application refers to a doctor at a hospital or emergency room providing remote support to an emergency medical technician (EMT).

Doctor (e.g., expert 22) is in communication with EMT (e.g., remote technician 20) via data network 12 and AR device 18. In particular embodiments, doctor 22 may receive video and audio from AR device 18 to help diagnose patient 24. Doctor 22 may annotate a whiteboard visible to EMT 20 via AR device 18 to assist EMT 20 with treatment of patient 24. Doctor 22 may project annotations onto patient 24 via AR device 18 to assist EMT 20 with treatment of patient 24. As EMT 20 repositions patient 24, the projected annotations may reposition with patient 24.

Although AR device 18 is illustrated as a handheld device, in particular embodiments AR device 18 may comprise a wearable device (e.g., AR glasses) to facilitate handsfree operation.

Although a particular example is illustrated in FIG. 2, particular embodiments are applicable to a variety of remote provisioning and diagnostic procedures. For example, in some embodiments an information technology (IT) specialist may remotely instruct a data center employee how to install and cable networking equipment. The IT specialist may annotate the locations of particular slots in a server shelf to install particular server blades and/or particular interface connections to connect particular cables.

Other examples may include services for do-it-yourself (DIY) homeowners. For example, a homeowner may request the services of an expert plumber when performing home plumbing repairs. The plumber may indicate which connections to remove/assemble, which tools to use, how to apply the tools, etc. In another example, a homeowner may request a furniture manufacturer to supply an expert to assist with furniture assembly. The expert may manipulate a CAD version of the furniture parts to illustrate assembly steps. The expert may annotate portions of the homeowner's furniture pieces to assist with assembly.

Although some examples are presented above, particular embodiments apply to other remote provisioning and diagnostic procedures.

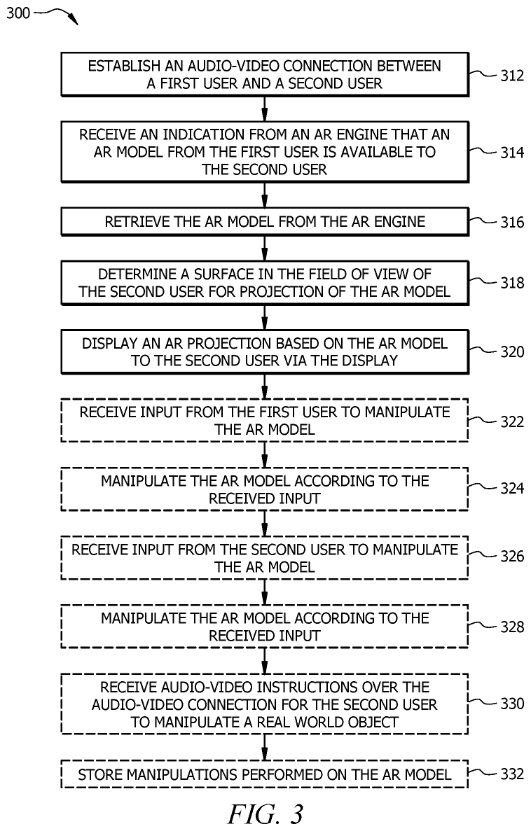

FIG. 3 illustrates a flowchart of a method performed by an AR device, in accordance with particular embodiments. In particular embodiments, one or more steps of FIG. 3 may be performed by an AR device described with respect to FIG. 4. The AR device comprises a display configured to overlay virtual objects onto a field of view of a user in real-time.

The method begins at step 312, where the AR device establishes an audio-video connection between a first user and a second user. For example, the AR device may establish an audio-video connection through AR engine 14. First user 22 may initiate the audio-video connection with second user 20, or second user 20 may initiate the audio-video connection with first user 22.

At step 314, the AR device receives an indication from an AR engine that an AR model from the first user is available to the second user. For example, as illustrated in FIG. 2, doctor 22 may provide an AR model to EMT 20 via data network 12 and AR engine 14. The AR model may comprise a whiteboard or an annotation of patient 24.

At step 316, the AR device retrieves the AR model from the AR engine. The AR model represents a real world object in the field of view of the second user. For example, AR device 18 may receive an AR model from AR engine 14.

At step 318, the AR device determines a surface in the field of view of the second user for projection of the AR model. For example, AR device 18 may determine the AR model should be projected on the torso or an appendage of patient 24. As another example, the surface may comprise a flat surface near patient 24. In other embodiments, the surface may comprise a surface of an object or device under repair.

At step 320, the AR device displays on the determined surface an AR projection based on the AR model to the second user via the display. The AR projection includes instructional information regarding the real world object. For example, AR device 18 may display AR projection 16 of patient 24 to EMT 20. AR projection 16 may include annotations indicating where a procedure should be performed on patient 24.

At step 322, the AR device may receive input from the first user to manipulate the AR model. For example, doctor 22 may send commands to AR engine 18 to manipulate the AR model. For example, doctor 22 may rotate or zoom in or out on particular area of patient 24. At step 324, the AR device may manipulate the AR model according to the received input. For example, AR device 18 manipulates AR projection 16 to display to EMT 20 the manipulations requested by doctor 22.

At step 326, the AR device may receive input from the second user to manipulate the AR model. For example, EMT 20 may rotate or zoom in or out on particular area of patient 24. At step 328, the AR device may manipulate the AR model according to the received input. For example, AR device 18 manipulates AR projection 16 to display to doctor 22 the manipulations requested by EMT 20. In particular embodiments, the AR device transmits the manipulations performed on the AR model by the second user to the first user.

At step 330, the AR model represents a real world object and AR device receives audio-video instructions over the audio-video connection for the second user to manipulate the real world object. For example, in coordination with any manipulations performed on the AR model, doctor 22 may also send audio and/or video instructions to EMT 20 for assisting patient 24.

At step 332, the AR device stores manipulations performed on the AR model. For example, AR device 18 may store manipulations for later review or audit. In particular embodiments, the AR device may also store any audio-video communications.

Modifications, additions, or omissions may be made to method 300 of FIG. 3. Additionally, one or more steps in the method of FIG. 3 may be performed in parallel or in any suitable order.

FIG. 4 is a block diagram illustrating an example augmented reality (AR) device. AR device 700 may be configured to overlay virtual content, according to any of the examples and embodiments described above. Examples of AR device 700 in operation are described with respect to FIGS. 1-3.

AR device 700 comprises a one or more processors 702, a memory 704, and a display 706. Particular embodiments may include a camera 708, a wireless communication interface 710, a network interface 712, a microphone 714, a global position system (GPS) sensor 716, and/or one or more biometric devices 718. AR device 700 may be configured as shown or in any other suitable configuration. For example, AR device 700 may comprise one or more additional components and/or one or more shown components may be omitted.

Processor 702 comprises one or more CPU chips, logic units, cores (e.g., a multi-core processor), FPGAs, ASICs, or DSPs. Processor 702 is communicatively coupled to and in signal communication with memory 704, display 706, camera 708, wireless communication interface 710, network interface 712, microphone 714, GPS sensor 716, and biometric devices 718. Processor 302 is configured to receive and transmit electrical signals among one or more of memory 704, display 706, camera 708, wireless communication interface 710, network interface 712, microphone 714, GPS sensor 716, and biometric devices 718. The electrical signals are used to send and receive data (e.g., images captured from camera 708, virtual objects to display on display 706, etc.) and/or to control or communicate with other devices. For example, processor 702 transmits electrical signals to operate camera 708. Processor 702 may be operably coupled to one or more other devices (not shown).

Processor 702 is configured to process data and may be implemented in hardware or software. Processor 702 is configured to implement various instructions and logic rules, such as instructions and logic rules 220. For example, processor 702 is configured to display virtual objects on display 706, detect hand gestures, identify virtual objects selected by a detected hand gesture (e.g., identify virtual content display opportunities), and capture biometric information of a user via one or more of camera 708, microphone 714, and/or biometric devices 718. In an embodiment, the functions of processor 702 may be implemented using logic units, FPGAs, ASICs, DSPs, or any other suitable hardware.

Memory 704 comprises one or more disks, tape drives, or solid-state drives, and may be used as an over-flow data storage device, to store programs when such programs are selected for execution, and to store instructions and data that are read during program execution, such as instructions and logic rules 220. Memory 704 may be volatile or non-volatile and may comprise ROM, RAM, TCAM, DRAM, and SRAM. Memory 704 is operable to store, for example, instructions for performing the functions of AR device 700 described herein, and any other data or instructions.

Display 706 is configured to present visual information to a user in an augmented reality environment that overlays virtual or graphical objects onto tangible objects in a real scene in real-time. In an embodiment, display 706 is a wearable optical display configured to reflect projected images and enables a user to see through the display. For example, display 706 may comprise display units, lens, semi-transparent mirrors embedded in an eye glass structure, a visor structure, or a helmet structure. Examples of display units include, but are not limited to, a cathode ray tube (CRT) display, a liquid crystal display (LCD), a liquid crystal on silicon (LCOS) display, a light emitting diode (LED) display, an active matrix OLED (AMOLED), an organic LED (OLED) display, a projector display, or any other suitable type of display as would be appreciated by one of ordinary skill in the art upon viewing this disclosure. In another embodiment, display 706 is a graphical display on a user device. For example, the graphical display may be the display of a tablet or smart phone configured to display an augmented reality environment with virtual or graphical objects overlaid onto tangible objects in a real scene in real-time.

Examples of camera 708 include, but are not limited to, charge-coupled device (CCD) cameras and complementary metal-oxide semiconductor (CMOS) cameras. Camera 708 is configured to capture images of a wearer of AR device 700, such as user 102. Camera 708 may be configured to capture images continuously, at predetermined intervals, or on-demand. For example, camera 708 may be configured to receive a command from user 102 to capture an image. In another example, camera 708 is configured to continuously capture images to form a video stream. Camera 708 is communicably coupled to processor 702.

Examples of wireless communication interface 710 include, but are not limited to, a Bluetooth interface, an RFID interface, an NFC interface, a local area network (LAN) interface, a personal area network (PAN) interface, a wide area network (WAN) interface, a Wi-Fi interface, a ZigBee interface, or any other suitable wireless communication interface as would be appreciated by one of ordinary skill in the art upon viewing this disclosure. Wireless communication interface 710 is configured to facilitate processor 702 to communicating with other devices. For example, wireless communication interface 710 is configured to enable processor 702 to send and receive signals with other devices. Wireless communication interface 710 is configured to employ any suitable communication protocol.

Network interface 712 is configured to enable wired and/or wireless communications and to communicate data through a network, system, and/or domain. For example, network interface 712 is configured for communication with a modem, a switch, a router, a bridge, a server, or a client. Processor 702 is configured to receive data using network interface 712 from a network or a remote source, such as cloud storage device 110, institution 122, mobile device 112, etc.

Microphone 714 is configured to capture audio signals (e.g. voice signals or commands) from a user, such as user 102. Microphone 714 is configured to capture audio signals continuously, at predetermined intervals, or on-demand. Microphone 714 is communicably coupled to processor 702.

GPS sensor 716 is configured to capture and to provide geographical location information. For example, GPS sensor 716 is configured to provide a geographic location of a user, such as user 28, employing AR device 700. GPS sensor 716 may be configured to provide the geographic location information as a relative geographic location or an absolute geographic location. GPS sensor 716 may provide the geographic location information using geographic coordinates (i.e., longitude and latitude) or any other suitable coordinate system. GPS sensor 716 is communicably coupled to processor 702.

Examples of biometric devices 718 include, but are not limited to, retina scanners and fingerprint scanners. Biometric devices 718 are configured to capture information about a person's physical characteristics and to output a biometric signal based on captured information. A biometric signal is a signal that is uniquely linked to a person based on their physical characteristics. For example, biometric device 718 may be configured to perform a retinal scan of the user's eye and to generate a biometric signal for the user based on the retinal scan. As another example, a biometric device 718 is configured to perform a fingerprint scan of the user's finger and to generate a biometric signal for the user based on the fingerprint scan. Biometric device 718 is communicably coupled to processor 702.

FIG. 5 illustrates an example of an apparatus to implement one or more example embodiments described herein. In this example, the apparatus 900 may include one or more processors 902, one or more output devices 905, and a memory 903. The apparatus 900 may be a computer.

In one embodiment, the one or more processors 902 may include a general purpose processor, an integrated circuit, a server, other programmable logic device, or any combination thereof. The processor may be a conventional processor, microprocessor, controller, microcontroller, or state machine. The one or more processors may be one, two, or more processors of the same or different types. Furthermore, the one or more processors may be a computer, computing device and user device, and the like.

In one example, based on user input 901 and/or other input from a computer network, the one or more processors 902 may execute instructions stored in memory 903 to perform one or more example embodiments described herein. Output produced by the one or more processors 902 executing the instructions may be output on the one or more output devices 905 and/or output to the computer network.

The memory 903 may be accessible by the one or more processors 902 via the link 904 so that the one or more processors 902 can read information from and write information to the memory 903. Memory 903 may be integral with or separate from the processors. Examples of the memory 903 include RAM, flash, ROM, EPROM, EEPROM, registers, disk storage, or any other form of storage medium. The memory 903 may store instructions that, when executed by the one or more processors 902, implement one or more embodiments of the invention. Memory 903 may be a non-transitory computer-readable medium that stores instructions, which when executed by a computer, cause the computer to perform one or more of the example methods discussed herein.

Numerous modifications, alterations, and changes to the described embodiments are possible without departing from the scope of the present invention defined in the claims. It is intended that the present invention is not limited to the described embodiments, but that it has the full scope defined by the language of the following claims, and equivalents thereof.

What is claimed is:

1. A system for performing remote instruction in an augmented reality (AR) environment, the system comprising an AR engine and an AR device;
the AR engine comprising one or more processors operable to:
establish an audio-video connection between a first user and a second user;
determine an AR device associated with the second user;
transmit an indication to the AR device that the AR model is available to the second user;
the AR device comprising a display configured to overlay virtual objects onto a field of view of the user in real-time and one or more processors coupled to the display, the one or more processors operable to:
receive the indication that the AR model is available to the second user;
retrieve the AR model from the AR engine;
determine a surface in the field of view of the second user for projection of the AR model; and
display on the determined surface an AR projection based on the AR model to the second user via the display.

2. The system of claim 1, the AR device one or more processors further operable to:
receive input from the first user to manipulate the AR model; and
manipulate the AR model according to the received input.

3. The system of claim 1, the AR device one or more processors further operable to:
receive input from the second user to manipulate the AR model; and
manipulate the AR model according to the received input.

4. The system of claim 3, wherein the AR device one or more processors are further operable to transmit the manipulations performed on the AR model by the second user to the first user.

5. The system of claim 1, wherein the AR model represents a real world object and the AR device one or more processors are further operable to receive audio-video instructions over the audio-video connection for the second user to manipulate the real world object.

6. The system of claim 1, wherein the AR engine one or more processors are further operable to store manipulations performed on the AR model.

7. The system of claim 1, wherein the first user comprises a medical expert and the second user comprises an emergency medical technician (EMT).

8. The system of claim 1, the first user comprises an expert repairman for a device and the second user comprises an owner of the device.

9. A method performed by an augmented reality (AR) device, the AR device comprising a display configured to overlay virtual objects onto a field of view of a user in real-time, the method comprising:
establishing an audio-video connection between a first user and a second user;
receiving an indication from an AR engine that an AR model from the first user is available to the second user;
retrieving the AR model from the AR engine, wherein the AR model represents a real world object in the field of view of the second user;
determining a surface in the field of view of the second user for projection of the AR model; and
displaying on the determined surface an AR projection based on the AR model to the second user via the display, wherein the AR projection includes instructional information regarding the real world object.

10. The method of claim 9, further comprising:
receiving input from the first user to manipulate the AR model; and
manipulating the AR model according to the received input.

11. The method of claim 9, further comprising:
receiving input from the second user to manipulate the AR model; and
manipulating the AR model according to the received input.

12. The method of claim 11, further comprising transmitting the manipulations performed on the AR model by the second user to the first user.

13. The system of claim 9, wherein the AR model represents a real world object and the method further comprises receiving audio-video instructions over the audio-video connection for the second user to manipulate the real world object.

14. The method of claim 10, further comprising storing manipulations performed on the AR model.

15. An augmented reality (AR) device, the AR device comprising a display configured to overlay virtual objects onto a field of view of a user in real-time and one or more processors coupled to the display, the one or more processors operable to:
establish an audio-video connection between a first user and a second user;
receive an indication from an AR engine that an AR model from the first user is available to the second user;
retrieve the AR model from the AR engine, wherein the AR model represents a real world object in the field of view of the second user;
determine a surface in the field of view of the second user for projection of the AR model; and
display on the determined surface an AR projection based on the AR model to the second user via the display, wherein the AR projection includes instructional information regarding the real world object.

16. The AR device of claim 15, the AR device one or more processors further operable to:
receive input from the first user to manipulate the AR model; and
manipulate the AR model according to the received input.

17. The AR device of claim 15, the AR device one or more processors further operable to:
receive input from the second user to manipulate the AR model; and
manipulate the AR model according to the received input.

18. The AR device of claim 17, the AR device one or more processors further operable to transmit the manipulations performed on the AR model by the second user to the AR engine.

19. The AR device of claim 15, wherein the AR model represents a real world object and the AR device one or more processors are further operable to receive audio-video instructions over the audio-video connection for the second user to manipulate the real world object.

20. The AR device of claim 15, the AR device one or more processors further operable to store manipulations performed on the AR model.
